(19) United States
(12) Patent Application Publication
(10) Pub. No.: US 2019/0224239 A1
(43) Pub. Date: Jul. 25, 2019

(54) HEAT-EXPOSED PLATELET LYSATE COMPOSITIONS, AND METHODS FOR PREPARING AND USING SAME

(71) Applicant: Cook Regentec LLC, Indianapolis, IN (US)

(72) Inventors: Jennifer Rodenberg, Battle Ground, IN (US); Eric Brandner, West Lafayette, IN (US); Hayley Steidinger, West Lafayette, IN (US); Amanda F. Taylor, West Lafayette, IN (US)

(73) Assignee: Cook Regentec LLC, Indianapolis, IN (US)

(21) Appl. No.: 16/254,995

(22) Filed: Jan. 23, 2019

Related U.S. Application Data
(60) Provisional application No. 62/620,515, filed on Jan. 23, 2018.

(51) Int. Cl. A61K 35/19; C12N 5/0783; A61K 38/48; A61K 38/57; A61K 38/20; A61K 38/18

(52) U.S. Cl. CPC A61K 35/19; C12N 2501/998; A61K 38/4886; A61K 38/57; A61K 38/2026; A61K 38/1825; A61K 38/1858; A61K 38/1866; A61K 38/1875; C12N 2502/115; C12N 2501/115; C12N 2501/165; C12N 2501/155; C12N 2501/135; C12N 2501/2304; C12N 2501/734; C12N 5/0646

(57) ABSTRACT

The present disclosure provides a composition derived from a platelet concentrate, methods of making the composition, and culture medium supplemented with the composition. Preferred methods of making the composition include heat treating a platelet lysate composition.

### BACKGROUND

The present invention relates generally to the field of bioactive materials derived from animal blood platelet products, and methods of preparation and use thereof.

The administration of cells or compositions containing cells for therapeutic treatment is becoming an increasingly popular treatment modality. Such treatments may include, for example, the administration of cells, such as immune cells and/or mesenchymal stem cells (MSCs) which have the potential to differentiate into mesenchymal lineage cells including, for instance, bone, fat, cartilage, and muscle.

In order to obtain therapeutic amounts of cells for transplant it is often necessary to expand a population of cells from an initial population. The culture media used to expand the cell population supplies essential nutrients for cell metabolism, growth, and proliferation. Fetal Bovine Serum (FBS) is often used as a supplement to encourage population expansion. FBS has been a preferred supplement due to its low level of antibodies, because it contains many growth factors which stimulate cell growth and proliferation, and because it is relatively inexpensive to manufacture. However, FBS has recognized disadvantages including the risk of transmission of pathogens such as bovine spongiform encephalopathy.

Human platelet lysate (hPL) has emerged as a potential non-xenogenic alternative to FBS. Platelet lysate is derived from platelets known to contain a variety of growth factors. In addition to growth factors, current hPL isolation techniques commonly result in compositions which retain a high concentration of fibrinogen, a glycoprotein involved in clot formation. Because of their fibrinogen content and tendency to clot, current commercial hPL compositions are often used in conjunction with one or more anticoagulant additives, typically heparin. Anticoagulant additives in hPL increase the cost of hPL production and/or use and may be problematic in situations where the bioactivity of heparin is detrimental. Also, while a variety of processes for producing hPL have been proposed, attempts to achieve target compositional profiles for the hPL product have often led to process complexity and/or intensive equipment requirements. Significant adoption of hPL as a substitute for FBS will require economically practicable processes which nonetheless yield desirable and effective compositions.

In view of this background, needs exist for human platelet lysate compositions having advantageous component profiles beneficial to cell proliferation, and that can be readily and economically manufactured.

### SUMMARY

In certain aspects of this disclosure, it has been discovered that bioactive platelet lysate compositions can be prepared to yield compositions having advantageous properties. The resulting compositions can have novel compositional profiles of growth factors and/or other substances present in the starting concentrates, and the processing methods can involve novel techniques for which alter the relative amounts of certain substances present in the starting material. Accordingly, in one embodiment, the present disclosure provides a method for preparing a bioactive platelet composition with modified bioactivity. The method comprises the steps of providing a starting platelet lysate composition including a first level of IL-4, and heating the platelet lysate composition under conditions and for a period of time effective to result in a modified platelet lysate composition including IL-4 at a level between 20% to 60% of the first level of IL-4. In accordance with certain inventive variants discussed herein the starting platelet lysate material has a reduced fibrinogen content, for example less than 20,000 ng/ml. In some forms the starting platelet lysate composition includes a first level of FGF-b, and the modified platelet lysate composition includes FGF-b at a second level of no more than 10% of the first level of FGF-b. In some forms the starting platelet lysate composition includes a first level of VEGF, and the modified platelet lysate composition includes VEGF at a second level of at least 65% of the first level of VEGF. In certain embodiments, the modified platelet lysate retains a percentage of other components form the starting material, for example: at least 40% of the starting TIMP-1, at least 70% of the starting PDGF-BB, at least 50% of the starting BMP-2, less than 10% of the starting MMP-1, less than 5% of the starting MMP-3, and/or 5% to 50% of the starting MMP-13. In some forms the bioactive platelet composition has a fibrinogen level of less than 20,000 ng/ml.

As stated above, in certain embodiments of the present disclosure a platelet lysate composition is heated under conditions and for a period of time effective to result in a modified platelet lysate composition. In accordance with certain inventive methods the heating step is performed for a duration of less than 1 hour. In some forms the heating step is performed for a less than 45 minutes. In accordance with certain inventive variants, the heating step may be performed at a temperature between about 55° C. to 65° C., preferably at temperatures between 58° C. to 62° C.

In another embodiment, the present disclosure provides a bioactive platelet lysate composition that has been heat treated under conditions and for a period of time effective to result in a modified platelet lysate composition having VEGF at a level of at least 200 pg/ml, IL-4 at a level of at least 30 pg/ml, and FGF-b at a level of less than 200 pg/ml. In certain embodiments, the modified platelet lysate retains a percentage of other components form the starting material, for example: at least 75,000 pg/ml TIMP-1, at least 7,500 pg/ml PDGF-BB, at least 10 pg/ml BMP-2, less than 300 pg/ml MMP-1, and/or less than 200 pg/ml MMP-3. In some forms the bioactive platelet composition has a fibrinogen level of less than 20,000 ng/ml.

In another embodiment, the present disclosure provides a platelet lysate composition having VEGF at a level of at least 200 pg/ml, FGF-b at a level of less than 200 pg/ml, and less than 20,000 ng/ml fibrinogen. In certain embodiments, the modified platelet lysate retains a percentage of other components form the starting material, for example: at least 75,000 pg/ml TIMP-1, at least 7,500 pg/ml PDGF-BB, at least 30 pg/ml IL-4, at least 10 pg/ml BMP-2, less than 300 pg/ml MMP-1, and/or less than 200 pg/ml MMP-3. In certain embodiments the bioactive composition includes FGF-b at a level of less than 100 pg/ml. In certain embodiments the bioactive composition includes FGF-b at a level of less than 10 pg/ml. In some forms, the bioactive composition of the present disclosure contains calcium at a level of 15-35 mg/dl.

Still further embodiments, as well as features and advantages, will be apparent to those of ordinary skill in the art from the descriptions herein.

### BRIEF DESCRIPTION OF THE DRAWINGS

FIG. 1 is a graph depicting the growth of the NK cells supplemented with various media over a 15-day period as detailed in Example 3.

FIG. 2 is a perspective view of one embodiment of a liquid bioactive fraction of the present disclosure in a sterile package.

FIG. 3 is a graph illustrating the relative concentration of active growth factors in one embodiment of the heat treated platelet lysate composition and a non-heat treated platelet lysate composition as detailed in Example 4.

FIG. 4a is a graph depicting the presence of active VEGF in platelet lysate compositions after subjection to heating for 0 minutes, 15 minutes, 30 minutes, 60 minutes, 2 hours, 4 hours, and 6 hours as detailed in Example 4.

FIG. 4b is a graph depicting the presence of active PDGF-AB in platelet lysate compositions after subjection to heating for 0 minutes, 15 minutes, 30 minutes, 60 minutes, 2 hours, 4 hours, and 6 hours as detailed in Example 4.

FIG. 4c is a graph depicting the presence of active TGF-β1 in platelet lysate compositions after subjection to heating for 0 minutes, 15 minutes, 30 minutes, 60 minutes, 2 hours, 4 hours, and 6 hours as detailed in Example 4.

FIGS. 4d and 4e are graphs depicting the presence of active FGF-basic in platelet lysate compositions after subjection to heating for 0 minutes, 15 minutes, 30 minutes, 60 minutes, 2 hours, 4 hours, and 6 hours as detailed in Example 4.

### DETAILED DESCRIPTION

For the purpose of promoting an understanding of the principles of the invention, reference will now be made to certain embodiments and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

In general the present disclosure provides bioactive compositions, and methods of making bioactive compositions, which can be used for example, as cell culture supplements and/or therapeutics. The compositions of the present disclosure include compositions derived from human sources, thus overcoming problems associated with xenogenic compositions such as fetal bovine serum (FBS). The discussions below in some respects expand upon options for each of these depicted general steps; however, it will be understood that not all of the depicted general steps are required for all embodiments herein, and that novel methods that include features corresponding to one, some, or all of the depicted steps are contemplated as embodiments herein.

As disclosed above, certain embodiments of the present disclosure relate to methods of making a novel platelet lysate composition having an advantageous component profile. In certain embodiments these methods include exposing a platelet composition to heat under conditions and for a period of time sufficient to degrade, denature, and/or inactivate certain components of the platelet composition. In some forms the platelet composition is a platelet lysate composition. As will be discussed herein the present disclosure also includes methods of use of a heat treated platelet composition for example in the preparation of a culture medium, in the culture of cells, or in the treatment of patients.

Platelet concentrate compositions used as source material for the disclosed methods and bioactive fractions may be obtained in any suitable way. As used herein, the term platelet concentrate refers to a liquid composition containing platelets that have been concentrated from a blood source. The blood source is preferably human blood, such as whole human peripheral blood. The platelet concentrate preferably includes both platelets and plasma proteins, and may be provided by platelet units obtained from whole peripheral blood of human donors by apheresis. It is envisioned that whole blood from other species, for example mammalian species, may also be used as a source for platelet concentrates to be processed as described herein. In certain embodiments, platelet units from different human or other donors can be pooled at some point during processing to obtain the bioactive fraction. In typical practice today, each human donor apheresed platelet unit has a volume of about 100 to about 500 ml, more typically about 100 to 400 ml, and contains about 100 to 500×10⁹ platelets along with plasma isolated with the platelets during the apheresis procedure. Donated human apheresis platelet units have a relatively brief shelf life for use at health care facilities, typically about five days. Platelet units used in methods disclosed herein can be recently expired human apheresis platelet units obtained from health care facilities, and can optionally be stored frozen at any suitable temperature, for example about −20° C., prior to use to prepare a bioactive fraction as described herein.

In preparing of a bioactive platelet composition, the contents of the platelets can be released by any suitable method. In some modes, the platelets are lysed by subjecting them to at least one freeze-thaw cycle to release the platelet contents, and optionally multiple freeze-thaw cycles (e.g. 2 or 3 freeze-thaw cycles). In use of a freeze-thaw cycle, a platelet concentrate can be frozen at any suitable temperature. In some aspects, the platelet concentrate is frozen at a temperature between about −10° C. and about −80° C. In specific preferred embodiments, the platelet concentrate is frozen at about −20° C. To lyse the platelets, the frozen platelet concentrate is thawed, for example in a 37 ° C. water bath or by other effective means, to form a “raw” platelet lysate composition. The raw platelet lysate contains the lysed platelet membranes, growth factors, fibrinogen, and other substances released from the lysed platelets. When the platelet concentrate being thawed contains plasma along with the platelets, the platelet lysate will also contain plasma, including plasma proteins therein. Other techniques for releasing platelet contents, for example activation with thrombin, may be used in certain aspects herein. However, freeze-thaw or other mechanical techniques for lysing the platelets are considered advantageous in that they do not require the addition of a non-native protein (e.g. thrombin) to the platelet concentrate, which addition both increases cost and leads to the presence of at least some of the thrombin in the downstream processed material. A “raw” platelet lysate as thus described can be subjected to heat treatment as described herein. However, in some forms the raw platelet lysate has been further processed to remove amounts of certain components, notably fibrinogen, prior to being subjected to heat treatment.

The raw platelet lysate contains multiple growth factors and other bioactive substances from the platelet concentrate starting material. These can include, for example, transforming growth factor beta 1, epidermal growth factor, basic fibroblast growth factor, platelet derived growth factor AA, platelet derived growth factor BB, platelet derived growth factor AB, stromal cell-derived factor-1α, vascular endothelial growth factor, metalloproteinase inhibitor 1, interleukin-4, and/or bone morphogenetic protein 2.

Transforming growth factor beta 1 (TGF-β1) is a multifunctional peptide that controls proliferation, differentiation, and other functions in many cell types. Epidermal growth factor (EGF) stimulates cellular proliferation, differentiation, and survival. Basic fibroblast growth factor (FGF-b) promotes angiogenesis, and binds to heparin which stimulates a wide variety of cells. Platelet derived growth factor AA (PDGF-AA) is a dimeric glycoprotein which regulates cell growth and division, and promotes angiogenesis. Platelet derived growth factor BB (PDGF-BB) is a dimeric glycoprotein which regulates cell growth and division, and promotes angiogenesis. Platelet derived growth factor AB (PDGF-AB) is a dimeric glycoprotein which regulates cell growth and division, and promotes angiogenesis. Stromal cell-derived factor-1α (SDF-1α) activates leukocytes and promotes angiogenesis. Vascular endothelial growth factor (VEGF) contributes to vasculogenesis and angiogenesis. Metalloproteinase inhibitor 1 (TIMP-1) is a glycoprotein that inhibits matrix metalloproteinases (MMPs). MMPs are a group of peptidases that contribute to the breakdown of extracellular matrix materials. Raw platelet lysate may also contain MMPs, including MMP-1, MMP-3, and MMP-13. Interleukin-4 (IL-4) is a cytokine with many known functions including activating both B-cell and T-cell proliferation, the differentiation of B cells into plasma cells, as well as regulating humoral and adaptive immune response. Bone morphogenetic protein 2 (BMP-2), along with other bone morphogenetic proteins, stimulates the production of bone and cartilage.

In certain embodiments, the raw platelet lysate includes the following amounts of growth factors and bioactive substances (based on the volume of original, undiluted platelet concentrate):

about 50,000 to about 150,000 pg/ml TGF-β1, preferably about 70,000 to about 120,000 pg/ml TGF-β1; and/or

about 100 to 600 pg/ml EGF, preferably about 200 to about 600 pg/ml EGF; and/or

about 5 to about 600 pg/ml FGF-b, preferably about 50 to 400 pg/ml FGF-b; and/or

about 500 to about 20,000 pg/ml PDGF-AA, preferably about 5000 to about 15000 pg/ml PDGF-AA; and/or

about 1000 to about 20,000 pg/ml PDGF-BB, preferably about 2000 to about 15000 pg/ml PDGF-BB; and/or

about 400 to 1100 pg/ml SDF-1α, preferably about 500 to about 1000 pg/ml SDF-1α; and/or

about 10 to about 800 pg/ml VEGF, preferably about 100 to about 600 pg/ml VEGF; and/or

about 50,000 to about 300,000 pg/ml TIMP-1, preferably about 100,000 to about 200,000 pg/ml TIMP-1; and/or

about 100 to about 500 pg/ml IL-4, preferably about 150-250 pg/ml IL-4; and/or

about 20 to about 100 pg/ml BMP-2, preferably about 40 to about 80 pg/ml BMP-2;

about 5,000 to about 20,000 pg/ml MMP-1, preferably about 8,000 to about 15,000 pg/ml MMP-1; and/or

about 5,000 to about 20,000 pg/ml MMP-3, preferably about 8,000 to about 15,000 pg/ml MMP-3; and/or

about 200 to about 1,000 pg/ml MMP-13, preferably about 400 to about 800 pg/ml MMP-13.

In preferred forms, the raw platelet lysate also includes one or more components derived from plasma in the platelet concentrate starting material, including for example fibrinogen, globulins, albumen, triglycerides, glucose, sodium, calcium, and/or cholesterol. In preferred forms, the raw platelet lysate includes the following components and amounts:

about 0.5 to 2.5 g/dL globulins, preferably about 1.5 to 2.5 g/dL globulins;

about 2 to 5 g/dL albumin, preferably about 3 to 4 g/dL albumin;

about 100 to 200 mmol/L sodium, preferably about 120 to about 160 mmol/L sodium;

about 40 to 200 mg/dL triglycerides, preferably about 50 to 120 mg/dL triglycerides;

about 150 to 300 mg/dL glucose, preferably about 150 to 250 mg/dL glucose;

about 5 to 12 mg/dL calcium, preferably about 6 to 10 mg/dL calcium; and/or

about 1 to 3.5 million ng/ml fibrinogen, preferably about 1.5 to 2.5 million ng/ml fibrinogen.

The raw platelet lysate can also contain other bioactive substances, for example one or more interleukins, interferons, and/or tumor necrosis factors. These interleukin(s), interferon(s) and/or tumor necrosis factor(s) may include, for example, one, some, or all of interleukin (IL)-1b, IL-6, IL-8, IL-10, IL-13, IL-17, interferon-gamma (IFN-gamma), and tumor necrosis factor-alpha (TNF-alpha).

In certain embodiments herein, the raw platelet lysate is processed to remove particulate matter, for example centrifuged, and sterilized for use as a platelet lysate product. Such sterilization can, for example, include passing the raw platelet lysate depleted of the particulate matter through a sterile filter, and/or irradiating the platelet lysate (e.g. e-beam radiation).

In some embodiments herein, the raw platelet lysate is treated to recover a fraction thereof with a reduced fibrinogen concentration. Fibrinogen may be removed by any suitable technique, including for example by conversion to fibrin resulting in the formation of solid clot material, which can be separated from a liquid bioactive fraction. Such conversion to fibrin can be induced by the addition of a clotting agent. In accordance with some forms of practicing the disclosed methods, a clotting agent, for example a calcium chloride salt, can be added to the raw platelet lysate. Illustratively, a calcium chloride salt can be added to the raw platelet lysate in an amount between about 0.1 g and 2 g per liter of raw platelet lysate. In preferred embodiments, about 0.4 g to about 0.7 g of a calcium chloride salt is added per liter of raw platelet lysate. The combined platelet lysate and calcium chloride or other clotting agent may be placed on a shaker or otherwise agitated to ensure thorough mixing of the clotting agent with the concentrate. The resulting mixture is then allowed to form a solid clot material, in certain embodiments for a period of at least about 8 hours, or at least about 12 hours, and typically in the range of about 8 hours to about 36 hours. In preferred forms, at least a predominant amount (over 50%) of the resulting clotted material, and potentially at least 80% or at least 90% of the resulting clotted material, is constituted by a substantially homogenous clot gel. Such a substantially homogenous clot gel can exhibit a consistent gel phase throughout the material, with liquid entrained within a continuous fibrin matrix. These preferred forms of clotted material are distinct from clotted platelet concentrate materials in which a multitude of discrete solid clot particles are suspended in a liquid phase, as would be desirable for a subsequent centrifuge-based separation technique.

After a solid clot has formed, liquid material can be separated from solid clot material. Any suitable technique may be used for this purpose. In preferred forms, the clotted material is pressed between two or more surfaces to separate clotted solids from liquid. In cases where the clotted material exhibits the form of a substantially homogenous clot gel as discussed herein, such pressing can express the liquid from the gel material while compressing and condensing the fibrin matrix of the gel. Pressing the clotted material can in some forms be conducted in a flexible container such as a plastic bag. The clot gel can be pressed, for example manually by hand or by forced application of an implement, to one region (e.g. end) of the bag or other flexible container and the liquid expressed from the solid fibrin matrix can gather in another region (e.g. end) of the bag or other flexible container. A second bag or other container can be connected to the first bag in which the pressing occurs, either during or after the pressing, and the liquid material can be transferred to the second bag or other container. In other modes, the clot gel can be in a rigid container such as a bucket, and can by pressed by hand or with the forced application of an implement to express the liquid from the solid fibrin matrix and compress and condense the fibrin matrix.

After clotting and separation of the liquid and solid materials of the clotted platelet concentrate, the separated liquid has a reduced concentration of fibrinogen as compared to the raw platelet lysate prior to clotting. In preferred forms, the raw platelet lysate has a fibrinogen content of at least one million ng/ml, typically in the range of about 1,500,000 to 3,500,000 (1.5 to 3.5 million) ng/ml, and after clotting and separation the liquid has a fibrinogen content of less than about 50,000 ng/ml, preferably less than about 20,000 ng/ml, and more preferably less than about 10,000 ng/ml. Illustratively, this separated liquid can have a fibrinogen content in the range of about 500 ng/ml to about 20,000 ng/ml, or about 500 ng/ml to about 10,000 ng/ml. Additionally or alternatively, this separated liquid can contain less than about 5% of the fibrinogen present in the platelet concentrate prior to clotting, preferably less than about 2%, and more preferably less than about 1%. As well, this separated liquid can constitute at least about 70% of the volume of the raw platelet lysate, preferably at least about 75%, and typically in the range of about 75% to about 90%.

As well, where a calcium chloride salt is used as a clotting agent for the raw platelet lysate, this separated liquid bioactive fraction can in some forms include calcium at a level of about 15 to 35 mg/dL, and preferably about 15 to 25 mg/dL.

The fibrinogen-depleted platelet lysate recovered after the clotting of the raw platelet lysate and the liquid solid/separation contains multiple growth factors from the raw platelet lysate. These can include TGF-β1, EGF, FGF-beta, PDGF-AA, PDGF-BB, SDF-1α, and VEGF. In certain embodiments, this fibrinogen-depleted liquid bioactive fraction includes the following growth factors and amounts thereof from the raw platelet lysate:

about 50,000 to about 175,000 pg/ml TGF-β1, preferably about 70,000 to about 120,000 pg/ml TGF-β1;

about 20 to 800 pg/ml EGF, preferably about 400 to about 800 pg/ml EGF; and/or

about 5 to about 350 pg/ml FGF-b, preferably about 50 to 250 pg/ml FGF-b; and/or

about 500 to about 25,000 pg/ml PDGF-AA, preferably about 5000 to about 18000 pg/ml PDGF-AA; and/or

about 1000 to about 25,000 pg/ml PDGF-BB, preferably about 2000 to about 18000 pg/ml PDGF-BB; and/or

about 400 to 1000 pg/ml SDF-1α, preferably about 500 to about 900 pg/ml SDF-1α; and/or

about 10 to about 650 pg/ml VEGF, preferably about 200 to about 500 pg/ml VEGF.

In other embodiments, the fibrinogen-depleted liquid bioactive fraction includes the following growth factors and amounts thereof from the raw platelet lysate:

FGF-2 (i.e. FGF-b) at a level of between about 200 pg/ml to about 350 pg/ml; and/or

EGF at a level of between about 1800 pg/ml to about 3100 pg/ml; and/or

PDGF-AA at a level of between about 24,000 pg/ml to about 28,000 pg/ml; and/or

PDGF-BB at a level of between about 50 ng/ml to about 80 ng/ml; and/or

VEGF at a level of between about 500 pg/ml to about 800 pg/ml; and/or

TGF-β1 at a level of between about 60 ng/ml to about 90 ng/ml.

In other embodiments, the fibrinogen-depleted liquid bioactive fraction includes the following growth factors and amounts thereof from the raw platelet lysate:

FGF-2 (i.e. FGF-b) at a level of between about 80 pg/ml to about 110 pg/ml, preferably about 90 to about 100 pg/ml; and/or

PDGF-AB at a level of between about 30,000 pg/ml to about 55,000 pg/ml, preferably about 35,000 to about 50,000 pg/ml; and/or

VEGF at a level of between about 400 pg/ml to about 800 pg/ml, preferably about 500 to about 700 pg/ml; and/or

TGF-β1 at a level of between about 150,000 pg/ml to about 200,000 pg/ml, preferably about 160,000 pg/ml to about 180,000 pg/ml.

In preferred forms, this fibrinogen-depleted liquid bioactive fraction also includes one or more components derived from plasma in the platelet concentrate starting material, including for example globulins, albumen, triglycerides, glucose, sodium, and/or calcium. Where a calcium chloride salt is used to clot the raw platelet lysate, the calcium present in the separated liquid bioactive agent can be from both the lysate and the added calcium salt. In certain embodiments, this separated liquid bioactive fraction includes the following components and amounts:

about 0.5 to 2.5 g/dL globulins, preferably about 1 to 2 g/dL globulins; and/or

about 2 to 5 g/dL albumin, preferably about 3 to 4 g/dL albumin; and/or

about 100 to 200 mmol/L sodium, preferably about 120 to about 160 mmol/L sodium; and/or

about 40 to 70 mg/dL triglycerides, preferably about 50 to 65 mg/dL triglycerides; and/or

about 150 to 300 mg/dL glucose, preferably about 150 to 250 mg/dL glucose; and/or

about 15 to 35 mg/dL calcium, and preferably about 15 to 25 mg/dL calcium.

In accordance with some modes of manufacture, the fibrinogen depleted platelet lysate is passed through a sterile filter prior to being heat treated as described herein. In preferred embodiments the sterile filter comprises a 0.2 μm sterile filter. Additionally or alternatively, the fibrinogen depleted platelet lysate can be subjected to other treatments prior to heating, including for example depth filtration and/or other pathogen reduction processes which can be orthogonal to the heat treatment, for example a solvent-detergent pathogen reduction process in which the platelet lysate is treated with a liquid medium containing a solvent and a detergent (e.g. a nonionic detergent), and or irradiation of the platelet lysate material.

In some embodiments the platelet lysate or a fraction thereof is heated. For example, during processing the platelet lysate material may be heated in a water bath or by another suitable means. As discussed herein, Applicants have surprisingly discovered that varying the temperature and duration of the heating procedure affects the component profile of the resulting heat treated material. In some forms, the platelet lysate material is heated to between about 50° C. to about 70° C. In preferred embodiments, the platelet lysate material is heated to between about 55° C. to about 65° C. In more preferred embodiments, the platelet lysate material is heated to between about 58° C. and about 62° C. In even more preferred embodiments, the platelet lysate material is heated to between about 58° C. and about 60° C. In accordance with certain embodiments, the platelet lysate material is kept at the preferred temperature for a predetermined time. In some forms, the platelet lysate material is held at the preferred temperature for about 15 minutes to about 6 hours. In preferred embodiments, the platelet lysate material is held at the preferred temperature for about 15 minutes to about 60 minutes. In certain embodiments the platelet lysate material is held at the preferred temperature for less than 2 hours. In preferred embodiments the platelet lysate material is held at the preferred temperature for less than 1 hour. In more preferred embodiments the platelet lysate material is held at the preferred temperature for less than 45 minutes. In some forms the platelet lysate material is held at the preferred temperature for at least 5 minutes, at least 10 minutes, or at least 15 minutes. In certain embodiments, the platelet lysate material is held at the preferred temperature for about 30 minutes. In certain embodiments, the platelet lysate material is held at the preferred temperature for about 60 minutes.

In certain embodiments, the heat treated platelet lysate includes the following amounts of active growth factors and bioactive substances:

about 1,000 to about 20,000 pg/ml PDGF-BB, preferably about 5,000 to about 15,000 pg/ml PDGF-BB, even more preferably about 10,000 to about 13,000 pg/ml PDGF-BB; and/or

about 5 to about 600 pg/ml FGF-b, preferably about 50 to 300 pg/ml FGF-b, even more preferably about 100 to about 250 pg/ml FGF-b; and/or

about 10 to about 800 pg/ml VEGF, preferably about 100 to about 600 pg/ml VEGF, even more preferably about 150 to about 300 pg/ml VEGF; and/or

about 25,000 to about 250,000 pg/ml TIMP-1, preferably about 50,000 to about 150,000 pg/ml TIMP-1; and/or

about 20 to about 200 pg/ml IL-4, preferably about 50 to about 150 pg/ml IL-4; and/or

about 20 to about 100 pg/ml BMP-2, preferably about 30 to about 70 pg/ml BMP-2;

about 50 to about 1,000 pg/ml MMP-1, preferably about 100 to about 500 pg/ml MMP-1, even more preferably about 100 to about 300 pg/ml MMP-1; and/or

about 25 to about 1,000 pg/ml MMP-3, preferably about 25 to about 500 pg/ml MMP-3, even more preferably about 25 to about 150 pg/ml MMP-3; and/or

about 50 to about 500 pg/ml MMP-13, preferably about 100 to about 500 pg/ml MMP-13, even more preferably about 100 to about 300 pg/ml MMP-13.

In certain embodiments, the heat treated platelet lysate includes the following amounts of active growth factors and bioactive substances:

about 25,000 to about 75,000 pg/ml PDGF-AB, preferably about 30,000 to about 60,000 pg/ml PDGF-AB, even more preferably about 35,000 to about 45,000 pg/ml PDGF-AB; and/or

about 0.1 to about 2 pg/ml FGF-b, preferably about 0.2 to about 1 pg/ml FGF-b, even more preferably about 0.5 to about 0.85 pg/ml FGF-b; and/or

about 300 to about 1,000 pg/ml VEGF, preferably about 400 to about 900 pg/ml VEGF, even more preferably about 500 to about 800 pg/ml VEGF; and/or

about 100,000 to about 200,000 pg/ml TGF-β1, preferably about 120,000 to about 180,000 pg/ml TGF-β1, even more preferably about 140,000 to about 160,000 pg/ml TGF-β1.

As discussed herein, heating of the platelet composition may cause degradation and/or inactivation of certain components. In some forms the level a specific bioactive component may be reduced by a certain percentage after heat treatment as compared with the level of the bioactive component prior to heat treatment. In certain embodiments, the heat treated platelet composition retains less than about 80% active TIMP-1 from the non-heat treated starting material, preferably less than about 70% active TIMP-1 from the non-heat treated starting material, even more preferably less than about 65% active TIMP-1 from the non-heat treated starting material. In certain embodiments the heat treated platelet lysate comprises TIMP-1 at a level of at least 40% that of a starting material prior to heat treatment. In a preferred embodiment the heat treated platelet lysate comprises TIMP-1 at a level of at least 50% that of a starting material prior to heat treatment. In even more preferred embodiments the heat treated platelet lysate comprises TIMP-1 at a level of at least 60% that of a starting material prior to heat treatment. In accordance with some forms the heat treated platelet lysate comprises TIMP-1 at a level of about 40% to about 80% that of a non-heat treated starting material prior to heat treatment, preferably about 50% to about 70% that of a non-heat treated starting material, even more preferably between about 60% to about 70% that of a non-heat treated starting material. In certain embodiments the heat treated platelet composition retains about 63% active TIMP-1 from a starting non-heat treated composition. Accordingly, the heat treated platelet composition may include at least 75,000 pg/ml active TIMP-1, preferably at least 90,000 pg/ml active TIMP-1, even more preferably at least 100,000 pg/ml active TIMP-1. In certain embodiments the heat treated platelet composition includes about 106,770.9 pg/ml active TIMP-1.

In certain embodiments, the heat treated platelet composition retains less than about 99% active PDGF-BB from the non-heat treated starting material, preferably less than about 98% active PDGF-BB from the non-heat treated starting material, even more preferably less than about 97% active PDGF-BB from the non-heat treated starting material. In certain embodiments the heat treated platelet lysate comprises PDGF-BB at a level of at least 70% that of a starting material prior to heat treatment. In a preferred embodiment the heat treated platelet lysate comprises PDGF-BB at a level of at least 80% that of a starting material prior to heat treatment. In even more preferred embodiments the heat treated platelet lysate comprises PDGF-BB at a level of at least 90% that of a starting material prior to heat treatment. In accordance with some forms the heat treated platelet lysate comprises PDGF-BB at a level of about 70% to about 90% that of a non-heat treated starting material prior to heat treatment, preferably about 80% to about 99% that of a non-heat treated starting material, even more preferably between about 90% to about 98% that of a non-heat treated starting material. In certain embodiments the heat treated platelet composition retains about 96% active PDGF-BB from a starting non-heat treated composition. Accordingly, the heat treated platelet composition may include at least 7,500 pg/ml active PDGF-BB, preferably at least 10,000 pg/ml active PDGF-BB, even more preferably at least 11,000 pg/ml active PDGF-BB. In certain embodiments the heat treated platelet composition includes about 11,800.7 pg/ml active PDGF-BB.

In certain embodiments, the heat treated platelet composition retains less than about 95% active VEGF from the non-heat treated starting material, preferably less than about 90% active VEGF from the non-heat treated starting material, even more preferably about 85% active VEGF from the non-heat treated starting material. In certain embodiments the heat treated platelet lysate comprises VEGF at a level of at least 65% that of a starting material prior to heat treatment. In a preferred embodiment the heat treated platelet lysate comprises VEGF at a level of at least 75% that of a starting material prior to heat treatment. In even more preferred embodiments the heat treated platelet lysate comprises VEGF at a level of at least 80% that of a starting material prior to heat treatment. In accordance with some forms the heat treated platelet lysate comprises VEGF at a level of about 65% to about 95% that of a non-heat treated starting material prior to heat treatment, preferably about 75% to about 90% that of a non-heat treated starting material, even more preferably between about 80% to about 90% that of a non-heat treated starting material. In certain embodiments the heat treated platelet composition retains about 85% active VEGF from a starting non-heat treated composition. In some forms the heat treated platelet lysate composition retains a substantial amount of the active VEGF from the non-heat treated starting material, for example at least 90%, more preferably at least 95%. Accordingly, the heat treated platelet composition may include at least 100 pg/ml active VEGF, preferably at least 150 pg/ml active VEGF, even more preferably at least 200 pg/ml active VEGF. In certain embodiments the heat treated platelet composition includes about 219.1 pg/ml active VEGF.

In certain embodiments, the heat treated platelet composition retains less than about 60% active IL-4 from the non-heat treated starting material, preferably less than about 50% active IL-4 from the non-heat treated starting material, even more preferably about 45% active IL-4 from the non-heat treated starting material. In certain embodiments the heat treated platelet lysate comprises IL-4 at a level of at least 20% that of a starting material prior to heat treatment. In a preferred embodiment the heat treated platelet lysate comprises IL-4 at a level of at least 30% that of a starting material prior to heat treatment. In even more preferred embodiments the heat treated platelet lysate comprises IL-4 at a level of at least 35% that of a starting material prior to heat treatment. In accordance with some forms the heat treated platelet lysate comprises IL-4 at a level of about 20% to about 60% that of a non-heat treated starting material prior to heat treatment, preferably about 30% to about 50% that of a non-heat treated starting material, even more preferably between about 35% to about 45% that of a non-heat treated starting material. In certain embodiments the heat treated platelet composition retains about 39% active IL-4 from a starting non-heat treated composition. Accordingly, the heat treated platelet composition may include at least 30 pg/ml active IL-4, preferably at least 60 pg/ml active IL-4, even more preferably at least 80 pg/ml active IL-4. In certain embodiments the heat treated platelet composition includes about 83.5 pg/ml active IL-4.

In certain embodiments, the heat treated platelet composition retains less than about 95% active BMP-2 from the non-heat treated starting material, preferably less than about 90% active BMP-2 from the non-heat treated starting material, even more preferably about 95% active BMP-2 from the non-heat treated starting material. In certain embodiments the heat treated platelet lysate comprises BMP-2 at a level of at least 50% that of a starting material prior to heat treatment. In a preferred embodiment the heat treated platelet lysate comprises BMP-2 at a level of at least 60% that of a starting material prior to heat treatment. In even more preferred embodiments the heat treated platelet lysate comprises BMP-2 at a level of at least 75% that of a starting material prior to heat treatment. In accordance with some forms the heat treated platelet lysate comprises BMP-2 at a level of about 50% to about 95% that of a non-heat treated starting material prior to heat treatment, preferably about 60% to about 90% that of a non-heat treated starting material, even more preferably between about 75% to about 85% that of a non-heat treated starting material. In certain embodiments the heat treated platelet composition retains about 83% active BMP-2 from a starting non-heat treated composition. Accordingly, the heat treated platelet composition may include at least 10 pg/ml active BMP-2, preferably at least 25 pg/ml active BMP-2, even more preferably at least 50 pg/ml active BMP-2. In certain embodiments the heat treated platelet composition includes about 54.0 pg/ml active BMP-2.

In certain embodiments, the heat treated platelet composition retains less than about 75% active FGF-b from the non-heat treated starting material, preferably less than about 65% active FGF-b from the non-heat treated starting material, even more preferably less than about 60% active FGF-b from the non-heat treated starting material. In certain embodiments the heat treated platelet lysate comprises FGF-b at a level of at least 25% that of a starting material prior to heat treatment. In some embodiments the heat treated platelet lysate comprises FGF-b at a level of at least 35% that of a starting material prior to heat treatment. In accordance with some forms the heat treated platelet lysate comprises FGF-b at a level of about 25% to about 75% that of a non-heat treated starting material prior to heat treatment, preferably about 35% to about 65% that of a non-heat treated starting material, even more preferably between about 50% to about 60% that of a non-heat treated starting material. In certain embodiments the heat treated platelet composition retains about 55% active FGF-b from a starting non-heat treated composition. Accordingly, the heat treated platelet composition may include 200 pg/ml or less active FGF-b, preferably 150 pg/ml or less active FGF-b, even more preferably 100 pg/ml or less active FGF-b. In certain embodiments the heat treated platelet composition includes about 182 pg/ml active FGF-b.

In accordance with certain embodiments the heat treated platelet lysate composition is rendered essential free of active FGF-b. Thus the heat treated platelet lysate of the present disclosure may have less than 2 pg/ml active FGF-b, preferably less than 1 pg/ml active FGF-b. The heat treated platelet lysate may retain less than about 10% active FGF-b from the non-heat treated starting material. The heat treated platelet lysate may retain less than about 1% active FGF-b from the non-heat treated starting material, preferably less than about 0.8% active FGF-b from the non-heat treated starting material, even more preferably less than about 0.5% active FGF-b from the non-heat treated starting material.

In certain embodiments, the heat treated platelet composition retains less than about 10% active MMP-1 from the non-heat treated starting material, preferably less than about 5% active MMP-1 from the non-heat treated starting material, even more preferably less than about 2% active MMP-1 from the non-heat treated starting material. In certain embodiments the heat treated platelet lysate comprises MMP-1 at a level of at least 0.1% that of a starting material prior to heat treatment. In a preferred embodiment the heat treated platelet lysate comprises MMP-1 at a level of at least 0.5% that of a starting material prior to heat treatment. In even more preferred embodiments the heat treated platelet lysate comprises MMP-1 at a level of at least 1% that of a starting material prior to heat treatment. In accordance with some forms the heat treated platelet lysate comprises MMP-1 at a level of about 0.1% to about 10% that of a non-heat treated starting material prior to heat treatment, preferably about 0.5% to about 5% that of a non-heat treated starting material, even more preferably between about 1% to about 2% that of a non-heat treated starting material. In certain embodiments the heat treated platelet composition retains about 1.6% active MMP-1 from a starting non-heat treated composition. Accordingly, the heat treated platelet composition may include 300 pg/ml or less active MMP-1, preferably 250 pg/ml or less active MMP-1, even more preferably 200 pg/ml or less active MMP-1. In certain embodiments the heat treated platelet composition includes about 189.5 pg/ml active MMP-1.

In certain embodiments, the heat treated platelet composition retains less than about 5% active MMP-3 from the non-heat treated starting material, preferably less than about 3% active MMP-3 from the non-heat treated starting material, even more preferably less than about 1% active MMP-3 from the non-heat treated starting material. In certain embodiments the heat treated platelet lysate comprises MMP-3 at a level of at least 0.1% that of a starting material prior to heat treatment. In a preferred embodiment the heat treated platelet lysate comprises MMP-3 at a level of at least 0.3% that of a starting material prior to heat treatment. In even more preferred embodiments the heat treated platelet lysate comprises MMP-3 at a level of at least 0.5% that of a starting material prior to heat treatment. In accordance with some forms the heat treated platelet lysate comprises MMP-3 at a level of about 0.1% to about 5% that of a non-heat treated starting material prior to heat treatment, preferably about 0.3% to about 3% that of a non-heat treated starting material, even more preferably between about 0.5% to about 1% that of a non-heat treated starting material. In certain embodiments the heat treated platelet composition retains about 0.6% active MMP-3 from a starting non-heat treated composition. Accordingly, the heat treated platelet composition may include 200 pg/ml or less active MMP-3, preferably 150 pg/ml or less active MMP-3, even more preferably 100 pg/ml or less active MMP-3. In certain embodiments the heat treated platelet composition includes about 78.6 pg/ml active MMP-3.

In certain embodiments, the heat treated platelet composition retains less than about 50% active MMP-13 from the non-heat treated starting material, preferably less than about 40% active MMP-13 from the non-heat treated starting material, even more preferably less than about 30% active MMP-13 from the non-heat treated starting material. In certain embodiments the heat treated platelet lysate comprises MMP-13 at a level of at least 5% that of a starting material prior to heat treatment. In a preferred embodiment the heat treated platelet lysate comprises MMP-13 at a level of at least 10% that of a starting material prior to heat treatment. In even more preferred embodiments the heat treated platelet lysate comprises MMP-13 at a level of at least 15% that of a starting material prior to heat treatment. In accordance with some forms the heat treated platelet lysate comprises MMP-13 at a level of about 5% to about 50% that of a non-heat treated starting material prior to heat treatment, preferably about 10% to about 40% that of a non-heat treated starting material, even more preferably between about 15% to about 30% that of a non-heat treated starting material. In certain embodiments the heat treated platelet composition retains about 23% active MMP-13 from a starting non-heat treated composition. Accordingly, the heat treated platelet composition may include at least 75 pg/ml active MMP-13, preferably at least 100 pg/ml active MMP-13, even more preferably at least 125 pg/ml active MMP-13. In certain embodiments the heat treated platelet composition includes about 144.7 pg/ml active MMP-13.

As noted above, in some embodiments of methods herein, the liquid bioactive fraction may be passed through at least one sterile filter. A variety of sterile filters and associated methods are known and can be used. Exemplary contaminants to be removed by the sterile filter(s) include, for example: staphyloccus aureus, pseudomonas aeruginosa, clostridium sporogenes, candida albicans, aspergillus niger, mycoplasma, and/or bacillus subtilis. The sterile filter(s) may be selected to exhibit relatively low protein binding. After sterile filtration, in preferred forms, the sterile filtered liquid bioactive fraction can have the same components as specified above for the depth filtered or other filtered liquid bioactive fraction, and also has levels of those components within the ranges specified above for the depth or other filtered liquid bioactive fraction. It will be understood, however, that some reduction in the levels of some or all of the components may occur during the sterile filtration.

Heat treatment of a platelet composition may occur at any point during processing of the material. For example, in some forms the platelet composition may be heat treated prior to lysis of the platelets. In some forms the composition may be heat treated after lysis of the platelets but before fibrinogen depletion. In certain preferred embodiments a fibrinogen depleted platelet lysate composition is heat treated after platelet lysis and fibrinogen depletion.

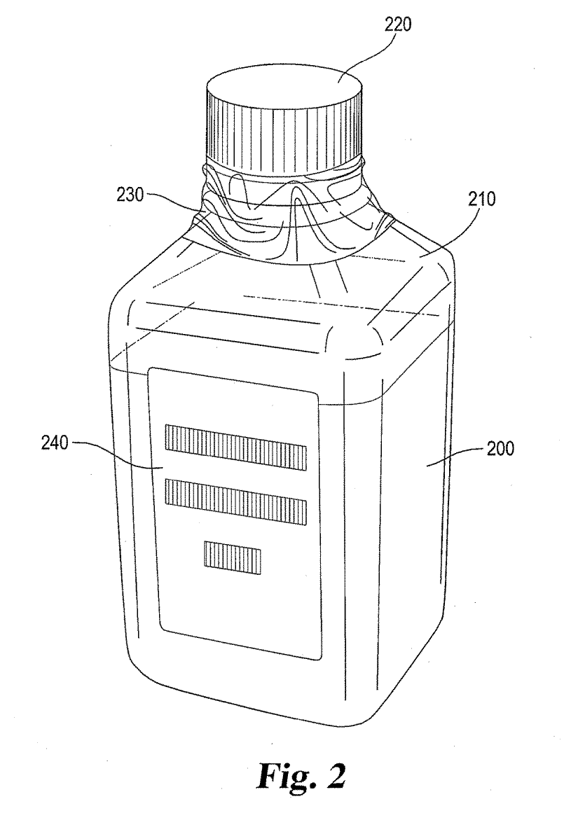

In some forms, bioactive platelet compositions of the present disclosure may be packaged in a sterile package for storage or delivery. The bioactive platelet fraction can be packaged at its full recovered concentration, or it may be diluted with water or an aqueous medium for packaging and later use, for example dilutions to 90% to 10% of the original concentration of the bioactive platelet composition can be prepared, and such diluted compositions, and their resulting corresponding reductions in the component levels specified herein, form additional embodiments disclosed herein. One embodiment of such packaging is illustrated in FIG. 2. In accordance with some forms of practicing the disclosure, the composition 200 is stored in a sterile media bottle 210. Sterile media bottles may, for example, have a volume capacity in the range of 50 ml to 5000 ml. As examples, 60 ml, 125 ml, 250 ml, 500 ml, 1000 ml, or 2000 ml bottles may be used. In some forms, cap 220 of sterile media bottle 210 is protected by shrink wrap 230. In some forms, the bottle is shrink wrapped. In certain embodiments, the bottle is labeled with a finished product label 240. In some forms, the bottle is placed in a product box with dry ice.

In certain embodiments, the bioactive platelet composition of the present disclosure may be combined with other ingredients to form a cell culture medium. Such a cell culture medium comprises the bioactive platelet composition of the present disclosure mixed with other nutrients or media for cell culture, including for example those as found in known cell culture media such as Minimum Essential Medium (MEM), or Dulbecco's Modified Eagle Medium (DMEM). A cell culture medium according to the present disclosure is formulated to provide nutrients (e.g. growth factors, etc.) necessary for the growth or maintenance of cells including for example stem and/or progenitor cells, such as mesenchymal stem cells, or immune cells such as T cells or natural killer (NK) cells. Cells cultured using the pathogen reduced platelet lysate or other platelet composition herein as or in a culture medium for the cells can in some embodiments be used medically by administering the cells to a human or other animal patient, and can have beneficial phenotypic and/or other characteristics for such administration. For example, mesenchymal or other stem cells can be administered to patients locally and/or systemically to the vasculature (e.g. by intravenous administration) for therapeutic purposes. Cultured immune cells, such as T cells, can be administered to patients to provide immunotherapy to treat cancer. In some forms, the immunotherapy is an adoptive cell transfer therapy in which a patients' own immune cells—collected from their blood or directly from their tumors—are to treat their cancer. The cancer can be melanoma, a blood cancer such as leukemia, or a lymphoma. In one mode of use, the heat treated platelet lysate is used in the culture of a patient's cells conjunction with “CAR T-Cell Therapy”, “TCR Therapy” or “TIL Therapy”.

In CAR T-cell therapy, a patients' T cells are collected from the blood, e.g. via apheresis. The T cells are then genetically modified to express a synthetic, or man-made, protein on their surface known as a chimeric antigen receptor, or CAR. The CARs on the T cells are designed to bind to specific proteins on the surface of cancer cells. After the T cells are engineered to express a CAR, they are then grown in the laboratory, using as or in the culture medium a heat treated platelet lysate as described herein, typically until there are over a hundred million of them. The CAR T cells are subsequently infused into the vascular system of the patient, usually after the patient has received chemotherapy and other drugs that deplete the body of existing T cells.

TCR therapy also involves engineering T cells collected from patients to express a receptor on their surface—called a T-cell receptor, or TCR. After the T cells are engineered to express a TCR, they are then grown, using as or in the culture medium a heat treated platelet lysate as described herein, typically until there are over a hundred million of them. The TCR engineered cells are subsequently infused into the vascular system of the patient, again usually after the patient has received chemotherapy and other drugs that deplete the body of existing T cells.

In TIL Therapy, tumor-infiltrating lymphocytes (TILs) are collected from a sample of a patient's tumor and tested to identify those with the greatest ability to recognize the patient's tumor cells. The identified TILs are then then grown, using as or in the culture medium a heat treated platelet lysate as described herein, typically until there are over a hundred million of them. The TIL cells are subsequently activated with cytokines and then infused into the vascular system of the patient, again usually after the patient has received chemotherapy and other drugs that deplete the body of existing T cells.

In some forms the bioactive platelet composition of the present disclosure can be used to supplement cell growth media. In particular a heat treated bioactive platelet composition of the present disclosure can be used as a cell growth supplement for the expansion of immune cells. In certain embodiments platelet compositions of the present disclosure may be used to as a cell growth substrate for the expansion of immune cells from peripheral blood mononuclear cells (PBMC), gene modified T cells, or Natural Killer cells (NK). In some forms the heat treated platelet lysate compositions of the present disclosure provide an advantageous component profile to support and sustain immune cell growth.

In other embodiments, the bioactive platelet composition of the present disclosure, or a fraction thereof, can be used as a therapeutic substance. For example, the composition can be used as a therapeutic substance for medical treatments, including for treatment of diseased or damaged tissue such as nerve, tendon, bone, muscle, skin (e.g. wound healing), connective, ocular and/or cardiovascular (e.g. heart or aorta) tissue. The bioactive platelet composition described herein or compositions including it can be delivered to these or other tissues by any suitable means including for example injection or other surgical implantation. In certain uses, in treating ocular tissue, the bioactive platelet composition or a composition including it is applied to the surface of an eye (e.g. in the form of liquid drops), for example in the treatment of ocular surface defects or diseases, such as ocular graft versus host disease (ocular GVHD), corneal ulcers, dry eye (Keratoconjunctivitis Sicca), or corneal repair after surgery or injury.

In accordance with certain forms, the platelet composition of the present disclosure is used to treat a mammalian patient (e.g. human, canine, feline, equine, etc.). In preferred modes of use, the platelet lysate or other platelet composition is allogeneic with respect to the treated patient, while in other embodiments the platelet lysate or other platelet composition can be xenogenic to with respect to the treated patient. For example, in certain embodiments, a heat treated platelet lysate composition derived from human platelets may be used to treat a canine patient. It is also envisioned that a heat treated platelet lysate composition derived from canine platelets may be used to treat a canine patient, and that a heat treated platelet lysate composition derived from human platelets may be used to treat a human patient. In some forms, the human patient is suffering from Keratoconjunctivitis Sicca. In accordance with certain inventive variants, a heat treated platelet lysate composition is used to treat a canine patient suffering from Keratoconjunctivitis Sicca. The canine patient may be any breed of canine, breeds commonly affected by Keratoconjunctivitis Sicca include: cavalier king charles spaniel, bulldog, Chinese shar-pei, Ihasa apso, shih tzu, west highland white terrier, pug, bloodhound, cocker spaniel, Pekingese, boston terrier, miniature schnauzer, and samoyed.

In accordance with other embodiments the heat treated platelet lysate or other platelet composition is formulated in an ointment. In some forms, the ointment is applied topically to an affected area (e.g. a patient's eye).

The bioactive platelet composition can also be used for other purposes, including for example as a cryopreservative for cells. In such cryopreservative uses, the bioactive platelet composition can be incorporated in a cellular suspension composition; the cellular suspension composition can be cryopreserved to preserve the viability of the cells. The cells can be any of a variety of cells, including stem cells such as mesenchymal stem cells, progenitor cells, or others. The cryopreservation can be conducted in a suitable vessel, such as a bag or vial.

In addition to deriving products from the recovered bioactive platelet fraction derived from the lysed platelet concentrate, valuable products can also be made from the solid clot material formed during the clotting and liquid-solid separation. In certain modes, the separated solid clot material has been discovered to also be rich in growth factors, and to contain sufficient amounts of fibrinogen and clotting factors to serve as a clottable vehicle, for example in biological adhesives, and/or to serve as a hemostatic material for medical applications. For these or other purposes, the recovered solid clot material can be stored in a refrigerated or frozen condition and/or can be lyophilized to form a dry material that can optionally be reduced to a powder form. For medical, diagnostic, research, or other applications, the solid clot material or fractions derived therefrom can be sterilized by any suitable means including for example by exposure to radiation or chemical sterilants (e.g. ethylene oxide).

As well, in addition to the recovery of the liquid bioactive fraction, and potentially also products made from the solid clot material formed during the clotting and liquid-solid separation discussed above, bioactive substances such as growth factors or other proteins can be recovered individually or in mixtures from the filter or filters used to process the platelet lysate. This can recover additional value from the original starting material. Illustratively, a filter used in filtering the platelet lysate composition, can thereafter be processed to recover one or more growth factors or other bioactive substances caught on the filter. This can be accomplished in any suitable manner. Illustratively, one or more proteins, such as growth factors, can be eluted from the filter by passage of an eluting liquid through the filter so as to overcome the attraction of the protein(s) to the filter media and thereby generate an eluate stream containing the protein(s). Where a charged (e.g. positively charged) filter is used, which retains proteins based at least in part on a charge interaction between the protein(s) and the charged filter media, the protein(s) may be recovered from the filter media by elution with salt solution(s), a change in the pH of the elution liquid (relative to that used during the initial filtration), or with an affinity elution medium (containing a ligand(s) for the protein(s) to be eluted). Gradient elution (e.g. with salt or pH gradients) may be used to sequentially elute fractions that are purified for or enriched in a specific protein or proteins of interest. The recovered protein or proteins may for example be any of those identified herein, preferably one or more of the growth factors, interleukins, interferons, and/or tumor necrosis factors identified herein. These may be used for example for therapeutic, diagnostic or research purposes. After recovery from the filtration media, they may optionally be purified and/or sterilized for these or other purposes.

For the purpose of promoting further understanding of aspects of the present disclosure and their features and advantages, the following specific examples are provided. It will be understood that these examples are illustrative, and not limiting, of embodiments of the present disclosure.

### EXAMPLES

### Example 1

### Preparation of Human Platelet Lysate Composition

Disease-screened apheresed human platelet units (obtained from peripheral blood) that had just expired after a 5-day shelf life are collected and frozen at −20° C. in a freezer until use. A number of the units (e.g. about 10 units) are removed from the freezer and thawed at room temperature, thus lysing the platelets and forming a “raw hPL” composition. The raw hPL from the selected units is pooled into a bag. Calcium chloride is added to the pooled raw hPL at a level of 0.7 grams/L (approximately 6 mM CaCl₂) and then thoroughly mixed with the raw hPL on a shaker at room temperature for 2 hours. After mixing, the CaCl₂-treated raw hPL is allowed to clot overnight at room temperature, during which time a firm, substantially homogeneous clotted gel mass forms from the volume of raw hPL.

While remaining closed, the bag containing the gel clot of raw hPL is manually pressed by hand to express liquid from the gel clot. This pressing is thoroughly done, resulting in a solid clot mass at one end of the bag and a separate liquid volume at the other end of the bag, adjacent an outlet spout. The separated liquid represents approximately 75-80% of the volume of the original, pooled raw hPL, and the solid clot material represents the remainder. The liquid is transferred from the bag to a second, refrigerated bag having a volume of 100 L. A sufficient number of such thaw-pool-clot-express runs are conducted to fill the refrigerated 100 L bag with liquid.

### Example 2

### Preparation of a Heat-Treated Platelet Lysate Composition

A platelet lysate composition prepared as described in Example 1 is obtained and placed in a storage container. The platelet lysate composition is placed in a water bath until the internal temperature of the platelet lysate composition reaches a target temperature of 58-60° C. Once the platelet lysate composition has reached the target temperature, it is kept at 58-60° C. for 30 minutes and removed from the water bath.

### Example 3

### Comparative Cell Culture of Immune Cells Supplemented With Heat Treated Platelet Lysate, Non-Heat Treated Platelet Lysate, and Human Serum

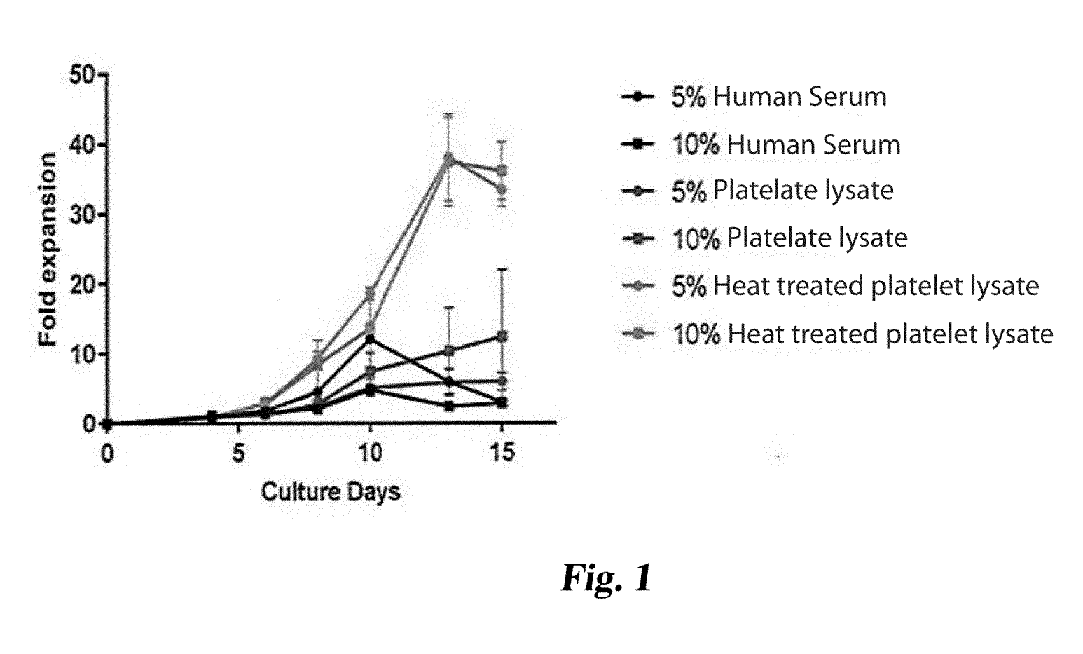

This test was performed to the growth of immune cells supplemented with heat treated platelet lysate, non-heat treated platelet lysate, and human serum. Natural Killer cells (NK cells) were cultured in media supplemented with one of the following: 5% human serum, 10% human serum, 5% platelet lysate, 10% platelet lysate, 5% heat treated platelet lysate, and 10% heat treated platelet lysate. Non-heat treated platelet lysate compositions were obtained as described in Example 1. Heat treated platelet lysate compositions were obtained as described in Example 2. FIG. 1 is a graph depicting the growth of the NK cells over a 15-day period.

### Example 4

### Growth Factor Analysis of Heat Treated Platelet Lysate

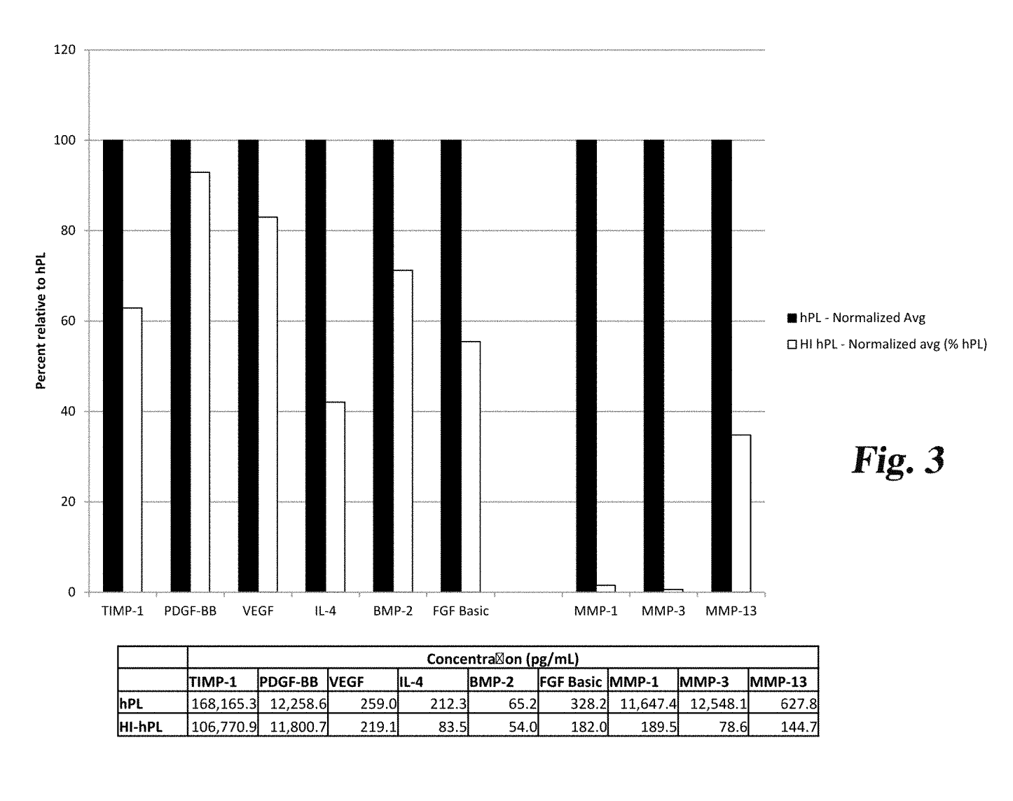

A comparative analysis was performed to compare the active growth factor profiles of platelet lysate compositions with and without heat treatment. Non-heat treated platelet lysate compositions were obtained as described in Example 1. Heat treated platelet lysate compositions were obtained as described in Example 2. Active growth factor measurements were performed for TIMP-1, PDGF-BB, VEGF, IL-4, BMP-2, FGF-b, MMP-1, MMP-3, and MMP-13 using ELISA kits and a microplate reader (Synergy Neo2 Plate Reader, BioTek Instruments). Growth factors for each treatment and control were analyzed in triplicate. ELISAs were performed according to manufacturer's protocol. Prior to running the ELISA, a dilution series was performed and appropriate dilutions were used for each growth factor analyzed. FIG. 3 is a graph illustrating the relative concentration of active growth factors in the heat treated platelet lysate composition and the non-heat treated platelet lysate composition. Table 1 shows the normalized average concentration (pg/ml) of the active growth factors in the non-heat treated platelet lysate composition and the heat treated platelet lysate composition.

TABLE 1

| | Non-Heat | Heat |
|---|---|---|
| | Treated | Treated |
| | Platelet | Platelet |
| | Lysate | Lysate |
| | (pg/ml) | (pg/ml) |
| TIMP-1 | 168,165.3 | 106,770.9 |
| PDGF-BB | 12,258.6 | 11,800.7 |
| VEGF | 259.0 | 219.1 |
| IL-4 | 212.3 | 83.5 |
| BMP-2 | 65.2 | 54.0 |
| FGF-b | 328.2 | 182.0 |
| MMP-1 | 11,647.4 | 189.5 |
| MMP-3 | 12,548.1 | 78.6 |
| MMP-13 | 627.8 | 144.7 |

Table 2 shows the percentage of active growth factors detected in the heat-treated platelet lysate compositions relative to the amount detected in the non-heat treated platelet lysate compositions.

TABLE 2

| | Percent of |
|---|---|
| | Growth factors |
| | in heat treated |
| | platelet lysate |
| | relative to |
| | non-heat treated |
| | platelet lysate |
| TIMP-1 | 63.5% |
| PDGF-BB | 96.3% |
| VEGF | 84.6% |
| IL-4 | 39.3% |
| BMP-2 | 82.8% |
| FGF-b | 55.5% |
| MMP-1 | 1.6% |
| MMP-3 | 0.6% |
| MMP-13 | 23.0% |

### Example 4

### Growth Factor Analysis of Heat Treated Platelet Lysate at Various Time Points

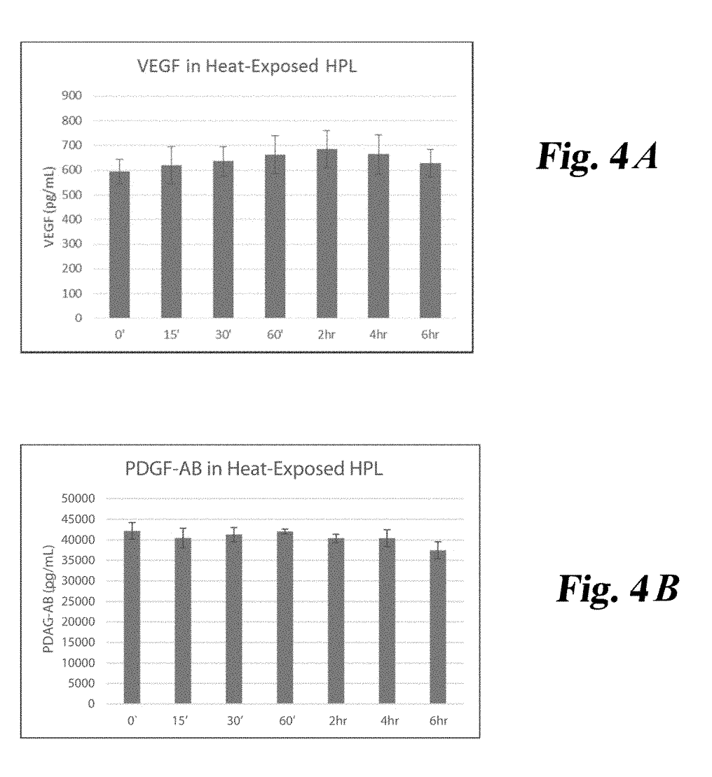

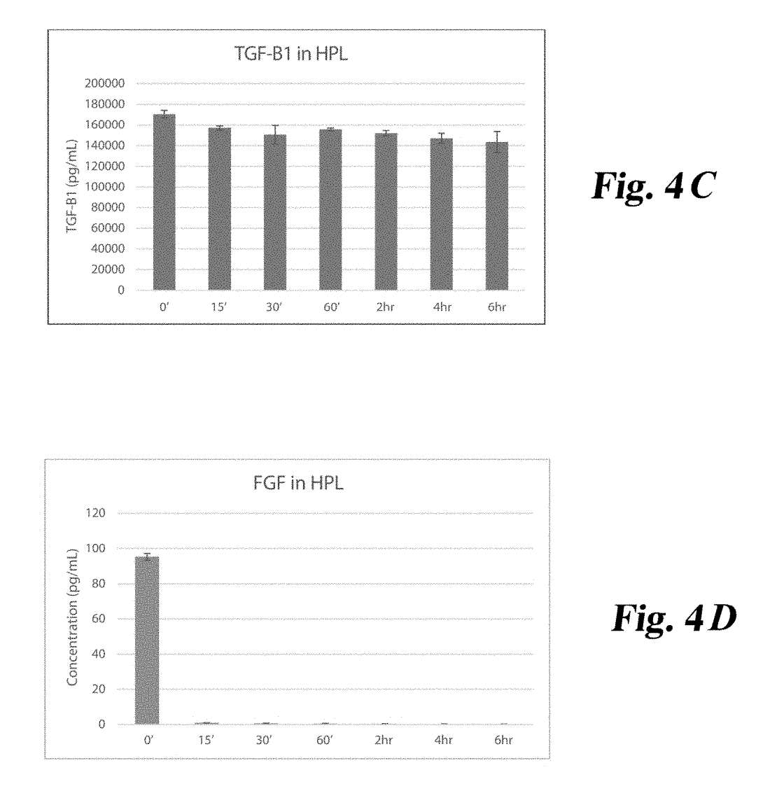

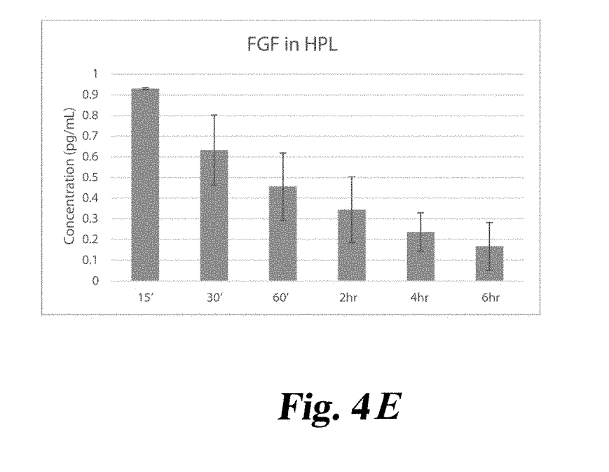

An analysis was performed of active growth factor concentration in platelet lysate compositions after subjection to heating for 0 minutes, 15 minutes, 30 minutes, 60 minutes, 2 hours, 4 hours, and 6 hours. A platelet lysate composition prepared as described in Example 1 was obtained and placed in a storage container. The platelet lysate composition was placed in a water bath until the internal temperature of the platelet lysate composition reaches a target temperature of 58-60° C. Once the platelet lysate composition had reached the target temperature, it was kept at 58-60° C. for the designated period of time. Active growth factor measurements were performed for VEGF, PDGF-AB, FGF-b, and TGF-β1 using ELISA kits and a microplate reader (Synergy Neo2 Plate Reader, BioTek Instruments). Growth factors for each treatment and control were analyzed in triplicate. ELISAs were performed according to manufacturer's protocol. Prior to running the ELISA, a dilution series was performed and appropriate dilutions were used for each growth factor analyzed. FIG. 4a depicts the results of the VEGF analysis. FIG. 4b depicts the results of the PDGF-AB analysis. FIG. 4c depicts the results of the TGF-β1 analysis. FIGS. 4d and 4e depict the results of the FGF-b analysis.

### Listing of Certain Embodiments

The following provides an enumerated listing of some of the embodiments disclosed herein. It will be understood that this listing is non-limiting, and that individual features or combinations of features (e.g. 2, 3 or 4 features) as described in the Detailed Description above can be incorporated with the below-listed Embodiments to provide additional disclosed embodiments herein.
1. A method for preparing a bioactive platelet composition with a modified bioactivity, the method comprising:

providing a starting platelet lysate composition including a first level of IL-4; and

heating the platelet lysate composition under conditions and for a period of time effective to result in a modified platelet lysate composition including IL-4 at a level of at least 20% of the first level of IL-4.
2. A method for preparing a bioactive platelet composition, the method comprising:

providing a starting platelet lysate composition, the starting platelet lysate composition having less than 20,000 ng/ml fibrinogen; and

heating the starting platelet lysate composition under conditions and for a period of time effective to modify a level of at least one growth factor.
3. The method of any one of embodiments 1 or 2, wherein the starting platelet lysate composition includes a first level of FGF-b, and wherein the modified platelet lysate composition includes FGF-b at a second level of no more than 10% of the first level of FGF-b.
4. The method of any one of embodiments 1 through 3, wherein the starting platelet lysate composition includes a first level of VEGF, and wherein the modified platelet lysate composition includes VEGF at a second level of at least 65% of the first level of VEGF.
5. The method of any one of embodiments 1 through 4, wherein the starting platelet lysate composition includes a first level of TIMP-1, and wherein the modified platelet lysate composition includes TIMP-1 at a second level of at least 40% of the first level of TIMP-1.
6. The method of any one of embodiments 1 through 5, wherein the starting platelet lysate composition includes a first level of PDGF-BB, and wherein the modified platelet lysate composition includes PDGF-BB at a second level of at least 70% of the first level of PDGF-BB.
7. The method of any one of embodiments 1 through 6, wherein the starting platelet lysate composition includes a first level of BMP-2, and wherein the modified platelet lysate composition includes BMP-2 at a second level of at least 50% of the first level of BMP-2.
8. The method of any one of embodiments 1 through 7, wherein the starting platelet lysate composition includes a first level of MMP-1, and wherein the modified platelet lysate composition includes MMP-1 at a second level of less than 10% of the first level of MMP-1.
9. The method of any one of embodiments 1 through 8, wherein the starting platelet lysate composition includes a first level of MMP-3, and wherein the modified platelet lysate composition includes MMP-3 at a second level of less than 5% of the first level of MMP-3.
10. The method of any one of embodiments 1 through 9, wherein the starting platelet lysate composition includes a first level of MMP-13, and wherein the modified platelet lysate composition includes MMP-13 at a second level of 5% to 50% of the first level of MMP-13.
11. The method of any one of embodiments 1 through 10, wherein fibrinogen is present at a level of less than 20,000 ng/ml in the starting platelet lysate composition.
12. The method of any one of embodiments 1 through 11, wherein said heating is performed for a duration of less than 1 hour.
13. The method of embodiment 12, wherein said heating is performed for a duration of less than 45 minutes.
14. The method of any one of embodiments 1 through 13, wherein said heating is performed at a temperature of between 55° C. to 65° C.
15. The method of embodiment 14, wherein said heating is performed at a temperature of between 58° C. to 62° C.
16. The method of any one of embodiments 1 through 15, including FGF-b at a second level of no more than 1% of the first level of FGF-b.
17. The method of any one of the preceding embodiments, wherein the starting platelet lysate composition includes a first level of TIMP-1, and wherein the modified platelet lysate composition includes TIMP-1 at a second level of 40% to 80% of the first level of TIMP-1.
18. The method of any one of the preceding embodiments, wherein the starting platelet lysate composition includes a first level of PDGF-BB, and wherein the modified platelet lysate composition includes PDGF-BB at a second level of 70% to 90% of the first level of PDGF-BB.
19. The method of any one of the preceding embodiments, wherein the starting platelet lysate composition includes a first level of VEGF, and wherein the modified platelet lysate composition includes VEGF at a second level of 65% to 95% of the first level of VEGF.
20. The method of any one of the preceding embodiments, wherein the starting platelet lysate composition includes a first level of BMP-2, and wherein the modified platelet lysate composition includes BMP-2 at a second level of 50% to 95% of the first level of BMP-2.
21. The method of any one of the preceding embodiments, wherein the starting platelet lysate composition includes a first level of MMP-1, and wherein the modified platelet lysate composition includes MMP-1 at a second level of 0.1% to 10% of the first level of MMP-1.
22. The method of any one of the preceding embodiments, wherein the starting platelet lysate composition includes a first level of MMP-3, and wherein the modified platelet lysate composition includes MMP-3 at a second level of 0.1% to 5% of the first level of MMP-3.
23. The method of any one of the preceding embodiments, wherein the modified platelet lysate composition includes TIMP-1 at a level of at least 75,000 pg/ml.
24. The method of any one of the preceding embodiments, wherein the modified platelet lysate composition includes PDGF-BB at a level of at least 7,500 pg/ml.
25. The method of any one of the preceding embodiments, wherein the modified platelet lysate composition includes VEGF at a level of at least 100 pg/ml.
26. The method of any one of the preceding embodiments, wherein the modified platelet lysate composition includes IL-4 at a level of at least 30 pg/ml.
27. The method of any one of the preceding embodiments, wherein the modified platelet lysate composition includes BMP-2 at a level of at least 10 pg/ml.
28. The method of any one of the preceding embodiments, wherein the modified platelet lysate composition includes MMP-1 at a level of 300 pg/ml or less.
29. The method of any one of the preceding embodiments, wherein the modified platelet lysate composition includes MMP-3 at a level of 200 pg/ml or less.
30. The method of any one of the preceding embodiments, wherein the modified platelet lysate composition includes MMP-13 at a level of at least 75 pg/ml.
31. The method of any one of the preceding embodiments, wherein the modified platelet lysate composition includes FGF-b at a level of 300 pg/ml or less.
32. The method of embodiment 31, wherein the modified platelet lysate composition includes FGF-b at a level of 200 pg/ml or less.
33. The method of embodiment 32, wherein the modified platelet lysate composition includes FGF-b at a level of 10 pg/ml or less.
34. The method of any one of embodiments 2 through 33 wherein the starting platelet lysate composition includes a first level of IL-4, and wherein the modified platelet lysate composition includes IL-4 at a second level between 20% to 60% of the first level of IL-4.
35. A bioactive platelet lysate composition comprising:

a platelet lysate composition that has been heat treated under conditions and for a period of time effective to result in a modified platelet lysate composition having VEGF at a level of at least 200 pg/ml, IL-4 at a level of at least 30 pg/ml, and FGF-b at a level of less than 200 pg/ml.
36. The bioactive platelet lysate composition of embodiment 35, wherein the modified platelet lysate composition retains TIMP-1 at a level of at least 75,000 pg/ml.
37. The bioactive platelet lysate composition of any one of embodiments 35 or 36, wherein the modified platelet lysate composition retains PDGF-BB at a level of at least 7,500 pg/ml.
38. The bioactive platelet lysate composition of any one of embodiments 35 through 37, wherein the modified platelet lysate composition retains BMP-2 at a level of at least 10 pg/ml.
39. The bioactive platelet lysate composition of any one of embodiments 35 through 38, wherein the modified platelet lysate composition includes MMP-1 at a level of less than 300 pg/ml.
40. The bioactive platelet lysate composition of any one of embodiments 35 through 39, wherein the modified platelet lysate composition includes MMP-3 at a level of less than 200 pg/ml.
41. The bioactive composition of embodiment 35, wherein the modified platelet lysate composition includes

about 0.5 to 2.5 g/dL globulins, preferably about 1 to 2 g/dL globulins;

about 2 to 5 g/dL albumin, preferably about 3 to 4 g/dL albumin;

about 100 to 200 mmol/L sodium, preferably about 120 to about 160 mmol/L sodium;

about 50 to 120 mg/dL triglycerides, preferably about 60 to 110 mg/dL triglycerides; and/or

about 150 to 300 mg/dL glucose, preferably about 150 to 250 mg/dL glucose.
42. The bioactive composition of any one of embodiments 35 through 41, wherein fibrinogen is present at a level of less than 20,000 ng/ml.
43. A cell growth supplement composition comprising:

the bioactive platelet lysate composition of any one of embodiments 35 through 42.
44. A method for culturing cells, comprising culturing a population of cells in a culture medium including the cell growth supplement of embodiment 43 or prepared using a method of any one of embodiments 1 through 34.
45. The method of embodiment 44, wherein the population of cells comprises natural killer cells.
46. A method of treating a patient, comprising administering to the patient a composition comprising the bioactive platelet lysate composition of any one of embodiments 35 to 42 or prepared using a method of any one of embodiments 1 through 34.
47. A platelet lysate composition comprising:

VEGF at a level of at least 200 pg/ml;

FGF-b at a level of less than 200 pg/ml; and

less than 20,000 ng/ml fibrinogen.
48. The platelet lysate composition of embodiment 47 further comprising, PDGF-BB at a level of at least 7,500 pg/ml.
49. The platelet lysate composition of any one of embodiments 47 or 48 further comprising, TIMP-1 at a level of at least 75,000 pg/ml.
50. The platelet lysate composition of any one of embodiments 47 through 49 further comprising, IL-4 at a level of at least 30 pg/ml.
51. The platelet lysate composition of any one of embodiments 47 through 50 further comprising, BMP-2 at a level of at least 10 pg/ml.
52. The platelet lysate composition of any one of embodiments 47 through 51 further comprising, MMP-1 at a level of 300 pg/ml or less.
53. The platelet lysate composition of any one of embodiments 47 through 52 further comprising, MMP-3 at a level of 200 pg/ml or less.
54. The platelet lysate composition of any one of embodiments 47 through 53 further comprising, MMP-13 at a level of at least 75 pg/ml.
55. The platelet lysate composition of any one of embodiments 47 through 54 having FGF-b at a level of 100 pg/ml or less.
56. The platelet lysate composition of embodiment 55 having FGF-b at a level of 10 pg/ml or less.
57. The platelet lysate composition of any one of embodiments 47 through 56, wherein the composition includes:

about 0.5 to 2.5 g/dL globulins, preferably about 1 to 2 g/dL globulins;

about 2 to 5 g/dL albumin, preferably about 3 to 4 g/dL albumin;

about 100 to 200 mmol/L sodium, preferably about 120 to about 160 mmol/L sodium;

about 50 to 120 mg/dL triglycerides, preferably about 60 to 110 mg/dL triglycerides; and/or

about 150 to 300 mg/dL glucose, preferably about 150 to 250 mg/dL glucose.
58. The platelet lysate composition of any one of embodiments 47 through 57, wherein the composition includes calcium at a level of 15-35 mg/dL.
59. The platelet lysate composition of any one of embodiments 35-42, wherein the composition includes calcium at a level of 15-35 mg/dL.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All publications and patent applications cited in this specification are herein incorporated by reference as if each individual publication or patent application were specifically and individually indicated to be incorporated by reference. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

1. A method for preparing a bioactive platelet composition, the method comprising:
providing a starting platelet lysate composition, the starting platelet lysate composition having less than 20,000 ng/ml fibrinogen; and
heating the starting platelet lysate composition under conditions and for a period of time effective to modify a level of at least one growth factor.

2. The method of claim 1, wherein the starting platelet lysate composition includes a first level of FGF-b, and wherein the modified platelet lysate composition includes FGF-b at a second level of no more than 10% of the first level of FGF-b.

3. The method of claim 1, wherein the starting platelet lysate composition includes a first level of VEGF, and wherein the modified platelet lysate composition includes VEGF at a second level of at least 65% of the first level of VEGF.

4. The method of claim 1, wherein the starting platelet lysate composition includes a first level of TIMP-1, and wherein the modified platelet lysate composition includes TIMP-1 at a second level of at least 40% of the first level of TIMP-1.

5. The method of claim 1, wherein the starting platelet lysate composition includes a first level of PDGF-BB, and wherein the modified platelet lysate composition includes PDGF-BB at a second level of at least 70% of the first level of PDGF-BB.

6. The method of claim 1, wherein the starting platelet lysate composition includes a first level of BMP-2, and wherein the modified platelet lysate composition includes BMP-2 at a second level of at least 50% of the first level of BMP-2.

7. The method of claim 1, wherein the starting platelet lysate composition includes a first level of MMP-1, and wherein the modified platelet lysate composition includes MMP-1 at a second level of less than 10% of the first level of MMP-1.

8. The method of claim 1, wherein the starting platelet lysate composition includes a first level of MMP-3, and wherein the modified platelet lysate composition includes MMP-3 at a second level of less than 5% of the first level of MMP-3.

9. The method of claim 1, wherein the starting platelet lysate composition includes a first level of MMP-13, and wherein the modified platelet lysate composition includes MMP-13 at a second level of 5% to 50% of the first level of MMP-13.

10. The method of claim 1, wherein said heating is performed for a duration of less than 1 hour.

11. The method of claim 1, wherein said heating is performed at a temperature of between 55° C. to 65° C.

12. The method of claim 1, wherein the modified platelet lysate composition includes TIMP-1 at a level of at least 75,000 pg/ml.

13. The method of claim 1, wherein the modified platelet lysate composition includes PDGF-BB at a level of at least 7,500 pg/ml.

14. The method of claim 1, wherein the modified platelet lysate composition includes VEGF at a level of at least 100 pg/ml.

15. The method of claim 1, wherein the modified platelet lysate composition includes IL-4 at a level of at least 30 pg/ml.

16. The method of claim 1, wherein the modified platelet lysate composition includes BMP-2 at a level of at least 10 pg/ml.

17. The method of claim 1, wherein the modified platelet lysate composition includes MMP-1 at a level of 300 pg/ml or less.

18. The method of claim 1, wherein the modified platelet lysate composition includes MMP-3 at a level of 200 pg/ml or less.

19. The method of claim 1, wherein the modified platelet lysate composition includes MMP-13 at a level of at least 75 pg/ml.

20. The method of claim 1, wherein the modified platelet lysate composition includes FGF-b at a level of 300 pg/ml or less.

21. The method of claim 1, wherein the starting platelet lysate composition includes a first level of IL-4, and wherein the modified platelet lysate composition includes IL-4 at a second level of at least 20% of the first level of IL-4.

22. A bioactive platelet lysate composition comprising:
a platelet lysate composition that has been heat treated under conditions and for a period of time effective to result in a modified platelet lysate composition having VEGF at a level of at least 200 pg/ml, IL-4 at a level of at least 30 pg/ml, and FGF-b at a level of less than 200 pg/ml.

23. The bioactive platelet lysate composition of claim 22, wherein the modified platelet lysate composition retains TIMP-1 at a level of at least 75,000 pg/ml.

24. The bioactive platelet lysate composition of claim 22, wherein the modified platelet lysate composition retains PDGF-BB at a level of at least 7,500 pg/ml.

25. The bioactive platelet lysate composition of of claim 22, wherein the modified platelet lysate composition retains BMP-2 at a level of at least 10 pg/ml.

26. The bioactive platelet lysate composition of claim 22, wherein the modified platelet lysate composition includes MMP-1 at a level of less than 300 pg/ml.

27. The bioactive platelet lysate composition of claim 22, wherein the modified platelet lysate composition includes MMP-3 at a level of less than 200 pg/ml.

28. The bioactive composition of claim 22, wherein the modified platelet lysate composition includes
about 0.5 to 2.5 g/dL globulins, preferably about 1 to 2 g/dL globulins;
about 2 to 5 g/dL albumin, preferably about 3 to 4 g/dL albumin;
about 100 to 200 mmol/L sodium, preferably about 120 to about 160 mmol/L sodium;
about 50 to 120 mg/dL triglycerides, preferably about 60 to 110 mg/dL triglycerides; and/or
about 150 to 300 mg/dL glucose, preferably about 150 to 250 mg/dL glucose.

29. The bioactive composition of claim 22, wherein fibrinogen is present at a level of less than 20,000 ng/ml.

30. The platelet lysate composition of claim 22, wherein the composition includes calcium at a level of 15-35 mg/dL.

31. A method for culturing cells, comprising culturing a population of cells in a culture medium including the cell growth supplement of claim 22.

32. The method of claim 31, wherein the population of cells comprises natural killer cells.

33. A method of treating a patient, comprising administering to the patient a composition comprising the bioactive platelet lysate composition of claim 22.

34. A platelet lysate composition comprising:
VEGF at a level of at least 200 pg/ml;
FGF-b at a level of less than 200 pg/ml; and
less than 20,000 ng/ml fibrinogen.

35. The platelet lysate composition of claim 34 further comprising, PDGF-BB at a level of at least 7,500 pg/ml.

36. The platelet lysate composition of claim 34 further comprising, TIMP-1 at a level of at least 75,000 pg/ml.

37. The platelet lysate composition of claim 34 further comprising, IL-4 at a level of at least 30 pg/ml.

38. The platelet lysate composition of claim 34 further comprising, BMP-2 at a level of at least 10 pg/ml.

39. The platelet lysate composition of claim 34 further comprising, MMP-1 at a level of 300 pg/ml or less.

40. The platelet lysate composition of claim 34 further comprising, MMP-3 at a level of 200 pg/ml or less.

41. The platelet lysate composition of claim 34 further comprising, MMP-13 at a level of at least 75 pg/ml.

42. The platelet lysate composition of claim 34 having FGF-b at a level of 100 pg/ml or less.

43. The platelet lysate composition of claim 34, wherein the composition includes:
about 0.5 to 2.5 g/dL globulins, preferably about 1 to 2 g/dL globulins;
about 2 to 5 g/dL albumin, preferably about 3 to 4 g/dL albumin;
about 100 to 200 mmol/L sodium, preferably about 120 to about 160 mmol/L sodium;
about 50 to 120 mg/dL triglycerides, preferably about 60 to 110 mg/dL triglycerides; and/or
about 150 to 300 mg/dL glucose, preferably about 150 to 250 mg/dL glucose.

44. The platelet lysate composition of claim 34, wherein the composition includes calcium at a level of 15-35 mg/dL.
